(19) United States
(12) Patent Application Publication
(10) Pub. No.: US 2023/0106556 A1
(43) Pub. Date: Apr. 6, 2023

(54) METHOD FOR PROVIDING INFORMATION ON MAJOR DEPRESSIVE DISORDERS AND DEVICE FOR PROVIDING INFORMATION ON MAJOR DEPRESSIVE DISORDERS BY USING SAME

(71) Applicant: BWAVE CORPORATION, Goyang-si, Gyeonggi-do (KR)

(72) Inventor: Seung Hwan LEE, Goyang-si Gyeonggi-do (KR)

(73) Assignee: BWAVE CORPORATION, Goyang-si, Gyeonggi-do (KR)

(21) Appl. No.: 17/914,351

(22) PCT Filed: Mar. 22, 2021
(86) PCT No.: PCT/KR2021/003534

§ 371 Date: Oct. 30, 2022

(30) Foreign Application Priority Data
Mar. 24, 2020 (KR) 10-2020-0035491

(51) Int. Cl. A61B 5/16; A61B 5/00; A61B 5/372; G16H 50/30

(52) U.S. Cl. CPC A61B 5/165; A61B 5/7267; A61B 5/372; A61B 5/725; G16H 50/30

(57) ABSTRACT

The present disclosure provides a method for providing information on a major depressive disorder, which is implemented by a processor, and a device using the same, the method comprising receiving brain wave data of an individual; generating brain activity data based on the brain wave data; and determining whether the individual's major depressive disorder is present by using a classification model configured to classify the major depressive disorder based on the brain activity data.

### BACKGROUND

### Technical Field

The present disclosure relates to a method for providing information on a major depressive disorder and a device for providing information on a major depressive disorder using the same, and more specifically, to a method for providing information on a major depressive disorder, which provides information on whether the major depressive disorder has occurred based on brain wave data, and a device for providing information on a major depressive disorder using the same.

### Related Art

Mental disorders may refer to dysfunction that appears in psychology or behavior. In this case, major depressive disorders may be caused by genetic causes, physical and temperamental causes, mental and psychological causes such as stress, and the like.

In particular, a major depressive disorder which is one of mental disorders, usually develops slowly and may exhibit symptoms of hyperactivity, a separation anxiety disorder, or intermittent depressive symptoms such as insomnia, sad feelings, preoccupation with the past, distraction, hopelessness, fatigue, and loss of appetite for many years.

In modern society, as a frequency of exposure to mental stress increases, a prevalence rate of major depressive disorders is increasing. However, major depressive disorders have many similar symptoms that are shared with other major depressive disorders, and degrees thereof vary from person to person, so that accurate distinguishment thereof may be difficult.

As such, developing optimal classification criteria for major depressive disorders may be important for an accurate diagnosis of the major depressive disorder.

Therefore, a development of new diagnostic criteria and systems for major depressive disorders allowing for an improvement in accuracy of diagnosis is continuously required.

### SUMMARY OF THE DISCLOSURE

Meanwhile, for a clear diagnosis of a major depressive disorder, functional magnetic resonance imaging (fMRI) based on dynamic neural activity, representing unique characteristics of each disorder, has emerged.

More specifically, according to fMRI analysis, patients with major depressive disorders may have different neural responses when reading emotional texts compared to normal individuals. Accordingly, a fMRI analysis result can be provided as information for an accurate diagnosis of major depressive disorders.

Meanwhile, in the fMRI, patients may complain of anxiety or fear. during a diagnosis process. Furthermore, the fMRI still has many limitations when applied to a diagnosis of major depressive disorders, such as involving expensive analysis costs, spatial and temporal restrictions, and the like.

In particular, the fMRI may have limitations to providing information with high reliability for an accurate diagnosis of a major depressive disorder because it only focuses on neural activity while processing emotional information and does not consider important pathologies such as altered cognitive processes.

Meanwhile, the inventors of the present disclosure have noted that, in relation to a major depressive disorder, changes in bio-signals will precede as part of a body's response.

In particular, the inventors of the present disclosure have paid attention to changes in brain wave data in relation to occurrence of a major depressive disorder, and were able to recognize that the limitations of the fMRI analysis described above can be overcome by a use of the brain wave data.

More specifically, the inventors of the present disclosure have recognized that features associated with major depressive disorders from brain wave signals can be extracted, and the major depressive disorders can be classified with higher reliability when using them.

As a result, the inventors of the present disclosure were able to develop a system for providing information on a major depressive disorder based on brain wave signals.

Meanwhile, the inventors of the present disclosure have recognized that an application of brain activity data of source activity which is activated together as well as brain wave data according to a specific stimulus, obtainable from a sensor of brain wave signals, can contribute to an accurate diagnosis of a major depressive disorder.

In particular, the inventors of the present disclosure were able to apply the brain activity data to the system for providing information in consideration of the fact that the brain activity data may reflect a functional neurological measure value.

Furthermore, in order to provide information with high reliability, the inventors of the present disclosure were able to apply a classification model which is learned by brain activity data and trained to predict a major depressive disorder to the system for providing information.

At this time, the inventors of the present disclosure tried to apply a classification model configured to determine whether a major depressive disorder has occurred in an individual based on main features that are highly associated with the major depressive disorder, to the system for providing information.

The inventors of the present disclosure were able to recognize that, as the main data is used, a problem of overfitting appearing in the model can be solved when feature parameters for all brain activity data are used.

As a result, it was confirmed that the inventors of the present disclosure can classify whether a major depressive disorder has occurred with high accuracy according to the application of the classification model. In particular, the inventors of the present disclosure have confirmed that, as a classification model configured to determine whether a major depressive disorder has occurred based on the main data is applied, it is possible to reflect cognitive characteristics according to individuals and to classify the major depressive disorder with high accuracy.

Accordingly, an object to be achieved by the present disclosure is to provide a method and device for providing information on a major depressive disorder, configured to determine whether a major depressive disorder has occurred in an individual by using brain wave data obtained from an individual, brain activity data, and further, a classification model.

Objects of the present disclosure are not limited to the objects described above, and other objects which are not mentioned will be clearly understood by those skilled in the art from the following description.

In order to solve the above problem, there is provided a method for providing information on a major depressive disorder according to an embodiment of the present disclosure. The method for providing information according to an embodiment of the present disclosure is a method for providing information on a major depressive disorder implemented by a processor, and the method includes receiving brain wave data of an individual; generating brain activity data based on the brain wave data; and determining whether the individual's major depressive disorder is present by using a classification model configured to classify the major depressive disorder based on the brain activity data.

According to a feature of the present disclosure, the method further includes, after generating the brain activity data, extracting a feature of the brain activity data, and the determining of whether the major depressive disorder is present may include determining whether the individual's major depressive disorder is present based on the feature, by using the classification model.

According to another feature of the present disclosure, the brain activity data is a plurality of pieces of brain activity data, and the extracting of the feature may include determining a functional connectivity between the plurality of pieces of brain activity data, and determining the feature of the brain activity data based on a network structural characteristic of the functional connectivity.

According to still another feature of the present disclosure, the determining of the functional connectivity may include determining a connectivity of a phase locking value (PLV) for each of the plurality of pieces of brain activity data. In addition, the determining of the feature of the brain activity data may include determining the feature based on strength and a clustering coefficient of the connectivity of the PLV for the each of the plurality of pieces of brain activity data.

According to still another feature of the present disclosure, the classification model may be further configured to output 0 or 1 depending on whether the individual's major depressive disorder is present. In this case, the determining of whether the major depressive disorder is present may include determining whether the individual's major depressive disorder is present according to an output result.

According to still another feature of the present disclosure, the brain activity data is a plurality of pieces of brain activity data, and the method further includes determining main data having a significant difference depending on whether the major depressive disorder is present among the plurality of pieces of brain activity data. In this case, the determining of whether the major depressive disorder is present may include determining whether the major depressive disorder is present based on the main data, by using the classification model.

According to still another feature of the present disclosure, the determining of the main data may include extracting a feature of each of the brain activity data; and determining the main data based on a statistical scoring method for the feature.

According to still another feature of the present disclosure, the main data may include brain activity data of a right isthmus of cingulate, and brain activity data of a left postcentral area.

According to still another feature of the present disclosure, the brain activity data of the right isthmus of cingulate may be at least one of theta strength, alpha strength, a theta clustering coefficient, and an alpha clustering coefficient. In addition, the brain activity data of the left postcentral area may be at least one of delta strength, alpha strength, and an alpha clustering coefficient.

According to still another feature of the present disclosure, the method may further include filtering the brain activity data based on a band pass filter, which is performed after the generating of the brain activity data.

According to still another feature of the present disclosure, the brain wave data may be defined as brain wave data obtained in a resting state.

According to still another feature of the present disclosure, the generating of the brain activity data may include converting the brain wave data into the brain activity data, by using at least one among low-resolution brain electromagnetic tomography (LORETA), standardized low-resolution brain electromagnetic tomography (sLORETA), exact resolution brain electromagnetic tomography (eLORETA), minimum-norm estimate (MNE) and dynamic statistical parametric mapping (dSPM).

According to still another feature of the present disclosure, the brain activity data may include source activity, or a current source density (CSD) in at least one brain area among banks of the superior temporal sulcus, caudal anterior cingulate, caudal middle frontal, cuneus, entorhinal, frontal pole, fusiform, inferior parietal, inferior temporal, insula, isthmus cingulate, lateral occipital, lateral orbito frontal, lingual, medial orbito frontal, middle temporal, para central, para hippocampal, pars opercularis, pars orbitalis, pars triangularis, pericalcarine, post central, posterior cingulate, precentral, precuneus, rostral anterior cingulate, rostral middle frontal, superior frontal, superior parietal, superior temporal, supramarginal, temporal pole, and transverse temporal.

According to still another feature of the present disclosure, the method may further include, when a risk of occurrence of the major depressive disorder for the individual is determined, according to a treatment progress, repeatedly performing the receiving of the brain wave data; the generating of the brain activity data; and the determining of whether the individual's major depressive disorder is present.

In order to solve the above problem, there is provided a device for providing information on a major depressive disorder according to another embodiment of the present disclosure. The device includes a receiver configured to receive brain wave data of an individual; and a processor coupled to the receiver to communicate therewith. In this case, the processor may be configured to generate brain activity data based on the brain wave data, and determine whether the individual's major depressive disorder is present by using a classification model configured to classify a major depressive disorder based on the brain activity data.

According to a feature of the present disclosure, the processor may be further configured to extract a feature of the brain activity data and determine whether the individual's major depressive disorder is present based on the feature, by using the classification model.

According to another feature of the present disclosure, the brain activity data may be a plurality of pieces of brain activity data. In this case, the processor may be further configured to determine a functional connectivity between the plurality of pieces of brain activity data and determine the feature of the brain activity data based on a network structural characteristic of the functional connectivity.

According to still another feature of the present disclosure, the processor may be further configured to determine a connectivity of a phase locking value (PLV) for each of the plurality of pieces of brain activity data and determine the feature based on strength and a clustering coefficient of the connectivity of the PLV for the each of the plurality of pieces of brain activity data.

According to still another feature of the present disclosure, the classification model may be further configured to output 0 or 1 depending on whether the individual's major depressive disorder is present, and the processor may be further configured to determine whether the individual's major depressive disorder is present according to an output result.

According to still another feature of the present disclosure, the brain activity data may be a plurality of pieces of brain activity data. In this case, the processor may be further configured to determine main data having a significant difference depending on whether the major depressive disorder is present among the plurality of pieces of brain activity data, and determine whether the major depressive disorder is present based on the main data, by using the classification model.

According to still another feature of the present disclosure, the processor may be further configured to extract a feature of each of the pieces of brain activity data and determine the main data based on a statistical scoring method for the feature.

According to still another feature of the present disclosure, the processor may be further configured to filter the brain activity data based on a band pass filter.

According to still another feature of the present disclosure, the brain wave data may be defined as brain wave data obtained in a resting state.

According to still another feature of the present disclosure, the processor may be configured to convert the brain wave data into the brain activity data, by using at least one among at least one among low-resolution brain electromagnetic tomography (LORETA), standardized low-resolution brain electromagnetic tomography (sLORETA), exact resolution brain electromagnetic tomography (eLORETA), minimum-norm estimate (MNE) and dynamic statistical parametric mapping (dSPM).

According to still another feature of the present disclosure, the brain activity data may include source activity, or a current source density (CSD) in at least one brain area among banks of the superior temporal sulcus, caudal anterior cingulate, caudal middle frontal, cuneus, entorhinal, frontal pole, fusiform, inferior parietal, inferior temporal, insula, isthmus cingulate, lateral occipital, lateral orbito frontal, lingual, medial orbito frontal, middle temporal, para central, para hippocampal, pars opercularis, pars orbitalis, pars triangularis, pericalcarine, post central, posterior cingulate, precentral, precuneus, rostral anterior cingulate, rostral middle frontal, superior frontal, superior parietal, superior temporal, supramarginal, temporal pole, and transverse temporal.

The details of other embodiments are included in the detailed description and drawings.

The present disclosure can contribute to a highly reliable diagnosis of a major depressive disorder by providing a system for providing information to which brain wave data according to a specific stimulus, obtainable from a sensor of brain wave signals, and further, brain activity data of source activity are applied.

Accordingly, the present disclosure can overcome limitations of an analysis method such as functional magnetic resonance imaging (fMRI) which provides low-reliable information since it does not consider important pathologies such as altered cognitive processes ad still has many limitations such as involving expensive analysis costs, spatial and temporal restrictions, and the like.

Furthermore, the present disclosure may provide information with high reliability on occurrence of a major depressive disorder by providing a system for providing information to which a classification model learned by brain wave data and/or brain activity data and trained to predict a major depressive disorder having a high-risk degree of occurrence is applied.

In particular, the present disclosure uses a classification model configured to determine whether a major depressive disorder has occurred in an individual on a basis of main data that is highly correlated with the major depressive disorder, so that it is possible to reflect cognitive characteristics according to individuals and allow for classification with high accuracy for a major depressive disorder.

Accordingly, users can easily obtain information on their own mental health without temporal and spatial restrictions. Moreover, since a medical team can obtain information on a suspected individual, continuous monitoring of the individual who is suspected of a major depressive disorder may be enabled.

Therefore, the present disclosure can contribute to an early diagnosis and a good treatment prognosis of a major depressive disorder by providing information on whether the major depressive disorder has occurred.

The effects according to the present disclosure are not limited by the contents exemplified above, and more various effects are included in the present disclosure.

### BRIEF DESCRIPTION OF THE DRAWINGS

FIG. 1A is a schematic diagram for explaining a system for providing information on a major depressive disorder using bio-signal data according to an embodiment of the present disclosure.

FIG. 1B is a schematic diagram for explaining a device for providing information on a major depressive disorder according to an embodiment of the present disclosure.

FIG. 1C is a schematic diagram illustrating a user mobile device receiving information from the device for providing information on a major depressive disorder according to an embodiment of the present disclosure.

FIG. 2 is a schematic flowchart for explaining a method of determining whether a major depressive disorder has occurred based on brain activity data of an individual in the device for providing information on a major depressive disorder according to an embodiment of the present disclosure.

FIG. 3A exemplarily illustrates a step in which brain wave data is received in the method for providing information on a major depressive disorder according to an embodiment of the present disclosure.

FIG. 3B exemplarily illustrates a step in which brain activity data is generated based on brain wave data in the method for providing information on a major depressive disorder according to an embodiment of the present disclosure.

FIG. 3C exemplarily illustrates a step in which features of brain activity data are extracted in the method for providing information on a major depressive disorder according to an embodiment of the present disclosure.

FIG. 3D exemplarily illustrates a step of determining whether an individual's major depressive disorder is present in the method for providing information on a major depressive disorder according to an embodiment of the present disclosure.

FIG. 4 illustrates an evaluation result of a classification model that is applied to the device for providing information on a major depressive disorder according to an embodiment of the present disclosure.

### DETAILED DESCRIPTION OF THE EMBODIMENT

Advantages and features of the present disclosure and methods to achieve them will become apparent from descriptions of embodiments herein below with reference to the accompanying drawings. However, the present disclosure is not limited to the embodiments disclosed herein but may be implemented in various different forms. The embodiments are provided to make the description of the present disclosure thorough and to fully convey the scope of the present disclosure to those skilled in the art. It is to be noted that the scope of the present disclosure is defined only by the claims. In connection with the description of drawings, the same or like reference numerals may be used for the same or like elements.

In the disclosure, expressions “have,” “may have,” “include” and “comprise,” or “may include” and “may comprise” used herein indicate presence of corresponding features (for example, elements such as numeric values, functions, operations, or components) and do not exclude the presence of additional features.

In the disclosure, expressions “A or B,” “at least one of A or/and B,” or “one or more of A or/and B,” and the like used herein may include any and all combinations of the associated listed items. For example, the “A or B,” “at least one of A and B,” or “at least one of A or B” may refer to all of case (1) where at least one A is included, case (2) where at least one B is included, or case (3) where both of at least one A and at least one B are included.

The expressions, such as “first,” “second,” and the like used herein, may refer to various elements, but do not limit the order and/or priority of the elements. Furthermore, such expressions may be used to distinguish one element from another element but do not limit the elements. For example, a first user device and a second user device indicate different user devices regardless of the order or priority. For example, without departing from the scope of the present disclosure, a first element may be referred to as a second element, and similarly, a second element may also be referred to as a first element.

It will be understood that when an element (for example, a first element) is referred to as being “(operatively or communicatively) coupled with/to” or “connected to” another element (for example, a second element), it can be understood as being directly coupled with/to or connected to another element or coupled with/to or connected to another element via an intervening element (for example, a third element). On the other hand, when an element (for example, a first element) is referred to as being “directly coupled with/to” or “directly connected to” another element (for example, a second element), it should be understood that there is no intervening element (for example, a third element).

According to the situation, the expression “configured to (or set to)” used herein may be interchangeably used with, for example, the expression “suitable for,” “having the capacity to,” “designed to,” “adapted to,” “made to,” or “capable of”. The term “configured to (or set to)” may not necessarily mean only “specifically designed to” in hardware. Instead, the expression “a device configured to” in any situation may mean that the device is “capable of operating together with another device or other components. For example, a “processor configured to (or set to) perform A, B, and C” may mean a dedicated processor (for example, an embedded processor) for performing a corresponding operation or a generic-purpose processor (for example, a central processing unit (CPU) or an application processor) which may perform corresponding operations by executing one or more software programs which are stored in a memory device.

Terms used in the present disclosure are used to describe specified embodiments of the present disclosure and are not intended to limit the scope of other embodiments. The terms of a singular form may include plural forms unless otherwise specified. All the terms used herein, which include technical or scientific terms, may have the same meaning that is generally understood by a person skilled in the art. It will be further understood that terms which are defined in a dictionary among terms used in the disclosure, can be interpreted as having the same or similar meanings as those in the relevant related art and should not be interpreted in an idealized or overly formal way, unless expressly defined in the present disclosure. In some cases, even in the case of terms which are defined in the specification, they cannot be interpreted to exclude embodiments of the present disclosure.

Features of various embodiments of the present disclosure may be partially or fully combined or coupled. As will be clearly appreciated by those skilled in the art, technically various interactions and operations are possible, and respective embodiments may be implemented independently of each other or may be implemented together in an associated relationship.

For clarity of interpretation of the present specification, terms used herein will be defined below.

The term “major depressive disorder” as used herein, is one of mood disorders and may refer to a mental disorder experiencing one or more major depressive episodes without a manic or hypomanic episode.

Meanwhile, in the present specification, major depressive disorders may include “major depressive disorder” and further, may include “depression”.

The term “brain wave data” as used herein may refer to an electroencephalogram (EEG) signal value recorded in a sensor that detects brain waves. More specifically, the brain wave data may be brain wave signals of a positive potential response appearing after a stimulus of a specific intensity. However, it is not limited thereto.

Meanwhile, as the brain wave data may be a signal or a signal value obtained from a sensor, it may be interpreted as the same meaning as sensor data in the present specification.

According to a feature of the present disclosure, the brain wave data may include brain wave data that is measured from at least one electrode among Fp1, Fp2, F3, Fz, F4, F8, T7, C3, C4, Cz, T8, P7, P3, Pz, P4, P8, O1, O2, FCz, TP9, TP10, Oz, AFz, F7, Fpz, AF7, AF3, AF4, AFB, F9, F5, F1, F2, F6, F10, FT9, FT7, FC5, FC3, FC1, FC2, FC4, FC6, FT8, FT10, C5, C1, C2, C6, TP7, CP5, CP3, CP1, CPz, CP2, CP4, CP6, TP8, P9, P5, P1, P2, P6, P10, PO9, PO7, PO3, POz, PO4, PO8, PO10, O9, Iz, O10, F11, F12, FT11, FT12, TP11, TP12, PO11, PO12, P11, P12, I11, I12 and IIz.

According to a feature of the present disclosure, the brain wave data may be brain wave data obtained in a resting state in which a stimulus is not applied to an individual, but is not limited thereto.

The term “brain activity data” as used herein may refer to data of source activity that is activated while a stimulus is output. In this case, the source activity may correspond to a current source density (CSD) for a brain active area.

For example, according to another feature of the present disclosure, the brain activity data may include source activity, or a current source density (CSD) in at least one brain area among banks of the superior temporal sulcus, caudal anterior cingulate, caudal middle frontal, cuneus, entorhinal, frontal pole, fusiform, inferior parietal, inferior temporal, insula, isthmus cingulate, lateral occipital, lateral orbito frontal, lingual, medial orbito frontal, middle temporal, para central, para hippocampal, pars opercularis, pars orbitalis, pars triangularis, pericalcarine, post central, posterior cingulate, precentral, precuneus, rostral anterior cingulate, rostral middle frontal, superior frontal, superior parietal, superior temporal, supramarginal, temporal pole, and transverse temporal. However, it is not limited thereto.

For example, the brain activity data may include brain activity data of a right isthmus of cingulate, and brain activity data of a left postcentral area.

As the brain activity data may be defined as source activity, it may be interpreted as having the same meaning as source data in the present specification.

Meanwhile, the brain activity data may be generated based on the brain wave data described above.

In addition, the brain activity data may be obtained by estimating source activity for a voxel corresponding to a source space, by using at least one among low-resolution brain electromagnetic tomography (LORETA), standardized low-resolution brain electromagnetic tomography (sLORETA), exact resolution brain electromagnetic tomography (eLORETA), minimum-norm estimate (MNE), weighted MNE (wMNE), dynamic statistical parametric mapping (dSPM), linearly constrained minimum variance (LCMV) beamformers programs—LORETA/sLORETA toolbox, brainstrom, eConnectome, fieldtrip and EEGlab.

Preferably, the brain activity data may be data obtained through wMNE, but is not limited thereto.

The term “main data” as used herein may refer to data (or features) that have a high contribution to classifying a major depressive disorder and normal.

According to a feature of the present disclosure, the main data may be determined by extracting features of the brain activity data and based on a statistical scoring method for the features.

For example, a connectivity of a phase locking value (PLV) for each of a plurality of pieces of brain activity data may be determined, and based on strength and a clustering coefficient of the connectivity of the PLV for each of a plurality of pieces of brain activity data, network indices that are features may be determined. Then, an independent sample t-test is performed on the determined feature, and features showing a significant difference depending on whether a major depressive disorder is present may be determined. Finally, after calculating Fisher's scores, main data having a high contribution to classifying a major depressive disorder may be determined by ordering them.

According to a feature of the present disclosure, the main data may include network indices for the right isthmus of cingulate and the left postcentral area.

In this case, the network index of the right isthmus of cingulate may be at least one of theta strength, alpha strength, a theta clustering coefficient, and an alpha clustering coefficient. In addition, the network index of the left postcentral area may be at least one of delta strength, alpha strength, and an alpha clustering coefficient. However, the main data is not limited thereto, and may be different depending on an individual.

The term “statistical scoring method” as used herein may refer to a scoring method for ordering according to a degree of association with a specific class (e.g., a major depressive disorder or normal) for class classification.

For example, the statistical scoring method may be performed by calculating Fisher's scores, which is one of methods for finding a minimum value of a function, or an independent sample t-test that can confirm a difference between two groups, but it is not limited thereto.

The term “classification model” used herein may refer to a model that is trained to classify a major depressive disorder based on an individual's brain wave data and/or brain activity data obtained from a brain wave measurement device and/or brain electromagnetic tomography.

According to a feature of the present disclosure, the classification model may be a model that is configured to extract a feature by taking main data as an input, and based on this, output a major depressive disorder or normal.

For example, the classification model may be further configured to output 0 or 1 depending on whether a major depressive disorder is present based on Equation 1 below.

$$Output = [ \frac{1}{n} {\sum }_{i = 1}^{n} max ( 0 , 1 - {y}_{i} ( ω \cdot {x}_{i - b} ) ) ] + λ {‖ ω ‖}^{2} \qquad [ Equation 1 ]$$

Hinge loss function, max(0,1−yᵢ(ω·x_{i-b}))
xᵢ=features for class 1 (MDD patients)
yᵢ=features for class 2 (healthy controls)
output 1 or 0 {1—true, 0—false}

Here, xᵢ is a feature value of an i-th major depressive disorder individual, yᵢ is a feature value of an i-th normal individual, ω refers to a weight, λ, refers to a regularization coefficient, and b refers to a constant. In this case, the constant b may be determined through calculation of a hyperplane.

Meanwhile, the classification model is not limited thereto and may be configured to output more various classes according to severity of the major depressive disorder.

The classification model may be a model based on at least one algorithm among a support vector machine (SVM), a decision tree, a random forest, an adaptive boosting (AdaBoost), and a penalized logistic regression (PLR). However, the classification model of the present disclosure is not limited thereto and may be based on more various learning algorithms.

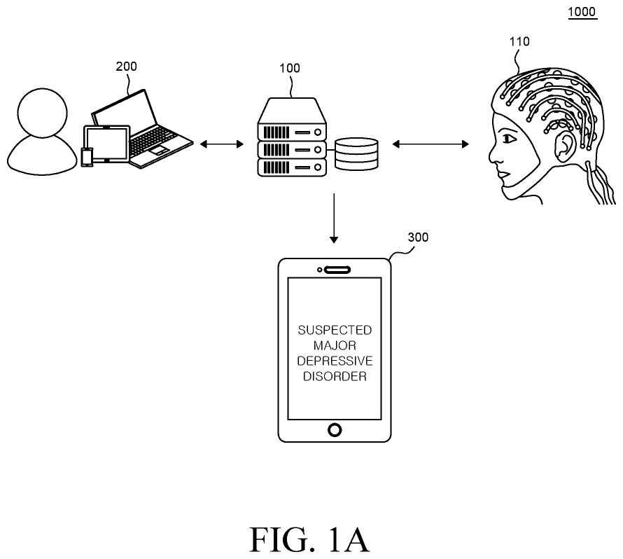

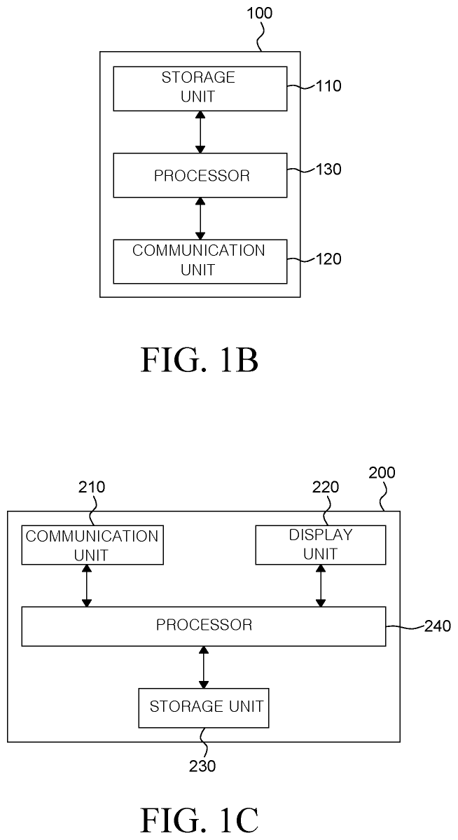

Hereinafter, a device for providing information on a major depressive disorder according to various embodiments of the present disclosure will be described in detail with reference to FIGS. 1A to 1C.

FIG. 1A is a schematic diagram for explaining a system for providing information on a major depressive disorder using bio-signal data according to an embodiment of the present disclosure.

First, referring to FIG. 1A, a system 1000 for providing information on a major depressive disorder may be a system that is configured to provide information related to a major depressive disorder based on a user's brain waves. At this time, the system 1000 for providing information on a major depressive disorder may be configured to include a device 100 for providing information on a major depressive disorder, which is configured to determine whether a major depressive disorder has occurred in an individual, based on the brain wave data and/or brain activity data, a user mobile device 200, a medical team device 300, and a brain wave measurement device 400 which is configured to be in close contact with a user's scalp and measure brain waves.

First, the device 100 for providing information on a major depressive disorder may include a general-purpose computer, a laptop, and/or a data server and the like that perform various operations to evaluate whether a major depressive disorder has occurred based on the user's brain wave provided from the brain wave measurement device 400. In this case, the user mobile device 200 may be a device for accessing a web server that provides a web page for a major depressive disorder or a mobile web server that provides a mobile website, but is limited thereto. Moreover, the brain wave measurement device 400 may be formed of a plurality of electrodes configured to cover the user's head from the outside. Meanwhile, the plurality of electrodes may include at least one standard electrode among Fp1, Fp2, F3, Fz, F4, F8, T7, C3, C4, Cz, T8, P7, P3, Pz, P4, P8, O1, O2, FCz, TP9, TP10, Oz, AFz, F7, Fpz, AF7, AF3, AF4, AFB, F9, F5, F1, F2, F6, F10, FT9, FT7, FC5, FC3, FC1, FC2, FC4, FC6, FT8, FT10, C5, C1, C2, C6, TP7, CP5, CP3, CP1, CPz, CP2, CP4, CP6, TP8, P9, P5, P1, P2, P6, P10, PO9, PO7, PO3, POz, PO4, PO8, PO10, O9, Iz, O10, F11, F12, FT11, FT12, TP11, TP12, PO11, PO12, P11, P12, I11, I12, and IIz.

Specifically, the device 100 for providing information on a major depressive disorder may be configured to receive brain wave data from the brain wave measurement device 400, extract features from the received brain wave data, and classify them as a major depressive disorder or normal.

The device 100 for providing information on a major depressive disorder may provide data which analyzes whether a major depressive disorder has occurred in an individual, to the user mobile device 200 and furthermore, to the medical team device 300.

As such, the data provided from the device 100 for providing information on a major depressive disorder may be provided as a web page through a web browser installed in the user mobile device 200 and/or the medical team device 300, or may be provided in a form of an application or program. In various embodiments, such data may be provided in a form included in a platform in a client-server environment.

Next, the user mobile device 200 is an electronic device that requests information on whether a major depressive disorder has occurred in the individual and provides a user interface for displaying analysis result data, and may include at least one of a smart phone, a tablet PC (a personal computer), a notebook computer and/or a PC and the like.

The user mobile device 200 may receive an analysis result on whether a major depressive disorder has occurred in the individual from the device 100 for providing information on a major depressive disorder, and display the received result through a display unit of the user mobile device 200. Here, the analysis result may include a risk degree of occurrence, a probability of occurrence and the like, of the major depressive disorder that is rated as high, medium, or low.

Next, referring to FIG. 1B, components of the device 100 for providing information on a major depressive disorder of the present disclosure will be described in detail.

FIG. 1B is a schematic diagram for explaining a device for providing information on a major depressive disorder according to an embodiment of the present disclosure.

Referring to FIG. 1B, the device 100 for providing information on a major depressive disorder includes a storage unit 110, a communication unit 120, and a processor 130.

First, the storage unit 110 may store various pieces of data for evaluating whether a major depressive disorder has occurred in an individual. In various embodiments, the storage unit 110 may include at least one type of storage medium among a flash memory type, a hard disk type, a multimedia card micro type, a card type memory (e.g., an SD or XD memory, etc.), a random-access memory (RAM), a static random-access memory (SRAM), a read-only memory (ROM), an electrically erasable programmable read-only memory (EEPROM), a programmable read-only memory (PROM), a magnetic memory, a magnetic disk, and an optical disk.

The communication unit 120 connects the device 100 for providing information on a major depressive disorder to an external device so that they can communicate with each other. The communication unit 120 may be connected to the user mobile device 200, the medical team device 300, and furthermore, the brain wave measurement device 400 using wired/wireless communication to transmit and receive various pieces of data. Specifically, the communication unit 120 may receive brain wave data of an individual from the brain wave measurement device 400, and may receive brain activity data from brain electromagnetic tomography (not shown). Also, the communication unit 120 may transmit the analysis result to the user mobile device 200 and/or the medical team device 300.

The processor 130 is operatively connected to the storage unit 110 and the communication unit 120, and may perform various commands for analyzing brain wave data and/or brain activity data for an individual.

Specifically, the processor 130 may receive the brain wave data of the individual from the brain wave measurement device 400 through the communication unit 120, generate brain activity data based on the received brain wave data, extract features therefrom and evaluate a risk degree of occurrence of the major depressive disorder for the individual.

Meanwhile, the processor 130 may be configured to convert the brain wave data into the brain activity data, using at least one among low-resolution brain electromagnetic tomography (LORETA), standardized low-resolution brain electromagnetic tomography (sLORETA), exact resolution brain electromagnetic tomography (eLORETA), minimum-norm estimate (MNE), weighted MNE (wMNE), dynamic statistical parametric mapping (dSPM), linearly constrained minimum variance (LCMV) beamformers programs—LORETA/sLORETA toolbox, brainstrom, eConnectome, fieldtrip and EEGlab.

Moreover, the processor 130 may be based on a classification model that is configured to classify whether a major depressive disorder has occurred based on the brain wave data and/or brain activity data.

In particular, the processor 130 may be based on a classification model that is configured to classify whether a major depressive disorder has occurred with higher reliability by using main data that is highly correlated with the major depressive disorder.

Accordingly, users can easily obtain information on their own mental health through the user mobile device 200 without temporal and spatial restrictions. Furthermore, since the medical team may obtain information on the individual from the medical team device 300, continuous monitoring of the individual who is suspected of a major depressive disorder may be enabled.

As described above, the present disclosure can contribute to an early diagnosis and a good treatment prognosis of a major depressive disorder by classifying whether the major depressive disorder has occurred with high accuracy and providing information thereon.

Meanwhile, referring to FIG. 1C, the user mobile device 200 includes a communication unit 210, a display unit 220, a storage unit 230, and a processor 240.

The communication unit 210 connects the user mobile device 200 to an external device so that they can communicate with each other. The communication unit 210 may be connected to the device 100 for providing information on a major depressive disorder using wired/wireless communication to transmit and receive various pieces of data. Specifically, the communication unit 210 may receive an analysis result related to a diagnosis of the major depressive disorder of an individual from the device 100 for providing information on a major depressive disorder.

The display unit 220 may display various interface screens for displaying analysis results related to the diagnosis of the major depressive disorder of the individual.

In various embodiments, the display unit 220 may include a touch screen and may receive, for example, a touch, gesture, proximity, drag, swipe, or hovering input or the like using an electronic pen or a body portion of the user.

The storage unit 230 may store various pieces of data used to provide a user interface for displaying result data. In various embodiments, the storage unit 230 may include at least one type of storage medium among a flash memory type, a hard disk type, a multimedia card micro type, a card type memory (e.g., an SD or XD memory, etc.), a random-access memory (RAM), a static random-access memory (SRAM), a read-only memory (ROM), an electrically erasable programmable read-only memory (EEPROM), a programmable read-only memory (PROM), a magnetic memory, a magnetic disk, and an optical disk.

The processor 240 is operatively connected to the communication unit 210, the display unit 220, and the storage unit 230, and may perform various commands for providing a user interface for displaying result data.

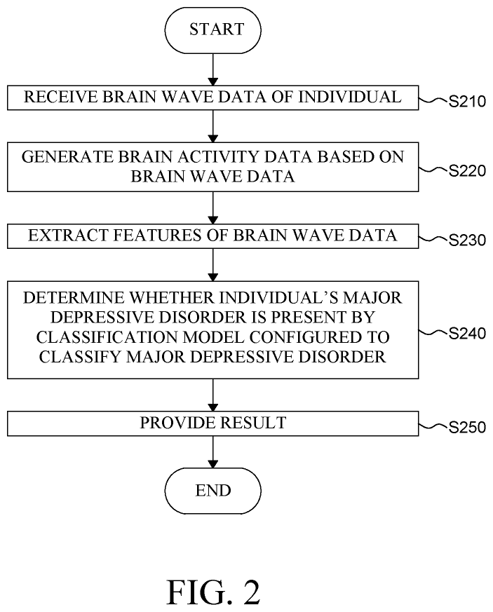

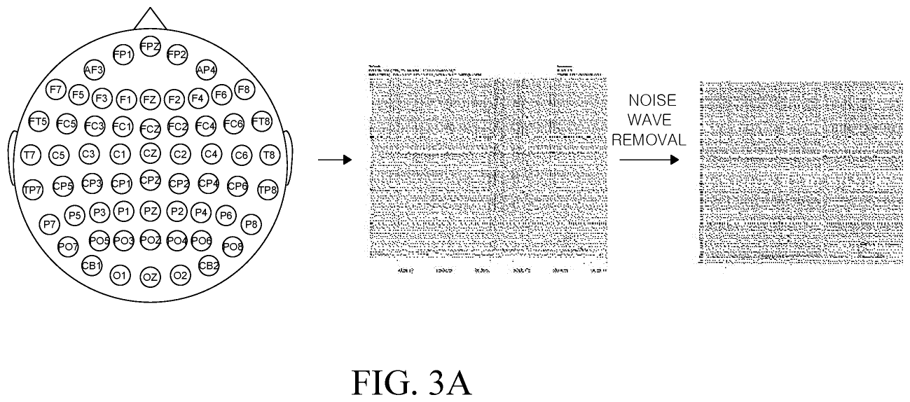

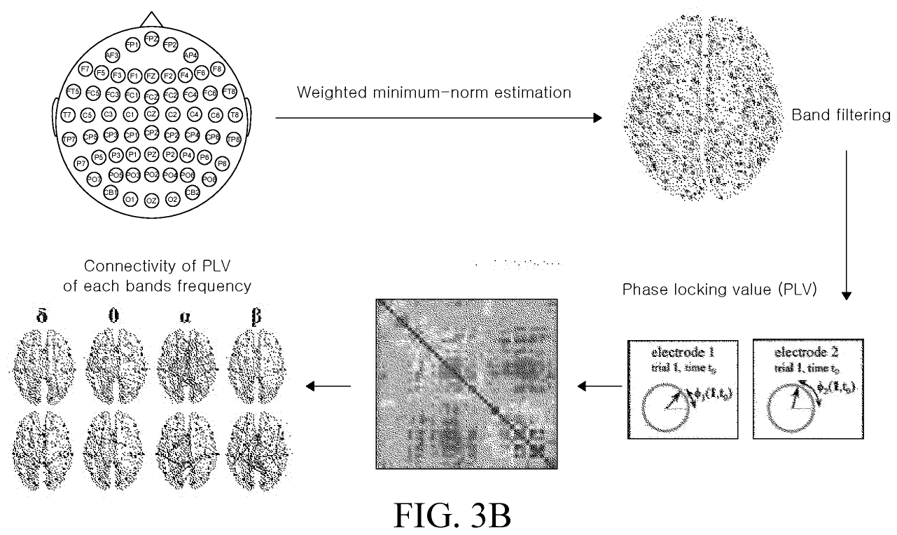

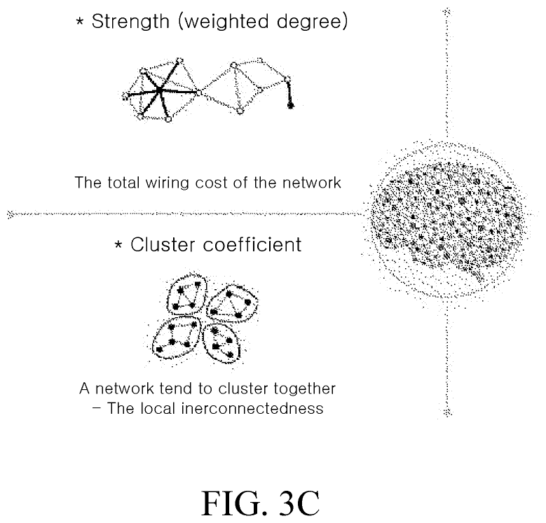

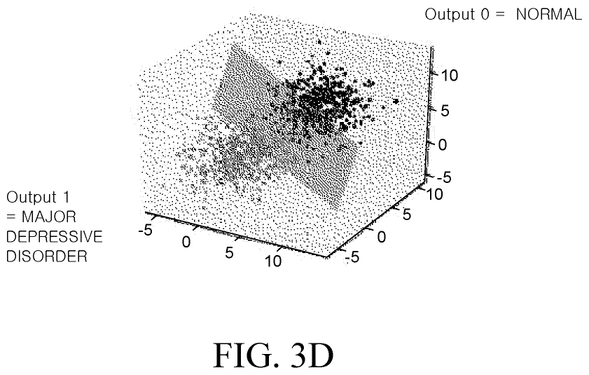

Hereinafter, a method for providing information according to various embodiments of the present disclosure will be described with reference to FIGS. 2 and 3A to 3D.

FIG. 2 is a schematic flowchart for explaining a method of determining whether a major depressive disorder has occurred based on brain activity data of an individual in the device for providing information on a major depressive disorder according to an embodiment of the present disclosure. FIG. 3A exemplarily illustrates a step in which brain wave data is received in the method for providing information on a major depressive disorder according to an embodiment of the present disclosure. FIG. 3B exemplarily illustrates a step in which brain activity data is generated based on brain wave data in the method for providing information on a major depressive disorder according to an embodiment of the present disclosure. FIG. 3C exemplarily illustrates a step in which features of brain activity data are extracted in the method for providing information on a major depressive disorder according to an embodiment of the present disclosure. FIG. 3D exemplarily illustrates a step in which whether an individual's major depressive disorder is present is determined in the method for providing information on a major depressive disorder according to an embodiment of the present disclosure.

First, referring to FIG. 2, brain wave data of an individual is received according to a method for providing information on a major depressive disorder according to an embodiment of the present disclosure, in step S210. Next, brain activity data is generated based on the brain wave data in step S220. Then, features are extracted from the brain activity data in step S230, and whether an individual's major depressive disorder is present is determined by a classification model in step S240. Finally, a final result is provided in step S250.

More specifically, in the step S210 in which the brain wave data of the individual is received, brain wave data obtained in a resting state may be obtained.

For example, referring together with FIG. 3A, in the step S210 in which the brain wave data of the individual is received, the brain wave data in a resting state can be obtained, which is measured from at least one electrode among Fp1, Fp2, F3, Fz, F4, F8, T7, C3, C4, Cz, T8, P7, P3, Pz, P4, P8, O1, O2, FCz, TP9, TP10, Oz, AFz, F7, Fpz, AF7, AF3, AF4, AFB, F9, F5, F1, F2, F6, F10, FT9, FT7, FC5, FC3, FC1, FC2, FC4, FC6, FT8, FT10, C5, C1, C2, C6, TP7, CP5, CP3, CP1, CPz, CP2, CP4, CP6, TP8, P9, P5, P1, P2, P6, P10, PO9, PO7, PO3, POz, PO4, PO8, PO10, O9, Iz, O10, F11, F12, FT11, FT12, TP11, TP12, PO11, PO12, P11, P12, I11, I12 and IIz. In this case, the brain wave data may be collected at 30 epochs at an interval of 2 seconds for the individual, but is not limited thereto. Meanwhile, when the brain wave data obtained in the step S210 in which the brain wave data of the individual is received includes noise waves, the noise waves may be removed.

Referring back to FIG. 2, brain activity data is generated based on the obtained brain wave data in step S220.

According to a feature of the present disclosure, in the step S220 in which the brain activity data is generated, the brain wave data may be converted into the brain activity data by at least one among low-resolution brain electromagnetic tomography (LORETA), standardized low-resolution brain electromagnetic tomography (sLORETA), exact resolution brain electromagnetic tomography (eLORETA), minimum-norm estimate (MNE), weighted MNE (wMNE), dynamic statistical parametric mapping (dSPM), linearly constrained minimum variance (LCMV) beamformers programs—LORETA/sLORETA toolbox, brainstrom, eConnectome, fieldtrip and EEGlab. Preferably, in the step S220 in which the brain activity data is generated, the brain activity data is generated by weighted MNE (wMNE).

According to another feature of the present disclosure, after the step S220 in which the brain activity data is generated, filtering may be performed on the generated brain activity data. That is, brain activity data for a specific frequency may be obtained by filtering.

For example, referring together with FIG. 3B, in the step S220 in which the brain activity data is generated, the brain activity data is generated by weighted MNE (wMNE), and then, brain activity data in a specific frequency area, more specifically, a delta wave (δ) of 1 to 4 Hz, a theta wave (θ) of 4 to 8 Hz, an alpha wave (α) of 8 to 12 Hz, and a beta wave (β) of 12 to 30 Hz may be obtained by a band pass filter.

Referring back to FIG. 2, in the step S230 in which features are extracted from the brain activity data, a network structural characteristic of the brain activity data may be determined.

According to a feature of the present disclosure, in the step S230 in which the features are extracted from the brain activity data, a functional connectivity between a plurality of pieces of brain activity data is determined, and based on a network structural characteristic of the functional connectivity, features of the brain activity data are determined.

According to another feature of the present disclosure, in the step S230 in which features are extracted, a connectivity of a phase locking value (PLV) for each of the plurality of pieces of brain activity data is determined, and based on strength and a clustering coefficient of the connectivity of the PLV for each of the plurality of pieces of brain activity data, the features may be extracted.

For example, referring back to FIG. 3B, in the step S230 in which features are extracted, with respect to the filtered brain activity data for a specific frequency, the phase locking value (PLV) may be calculated, which is a synchrony value for examining a change induced in a work in long-distance synchronization of neural activity. As a result, a functional connectivity matrix is formed, and the connectivity of the PLV of each bands frequency (δ, θ, α, or β) is determined. Next, referring to FIG. 3C, in the step S230 in which the features are extracted from the brain activity data, strength corresponding to a total wiring cost of the connectivity of the PLV is calculated, and/or a clustering coefficient corresponding to a cluster tendency for the connectivity of the PLV is calculated.

That is, as a result of the step S230 in which the features are extracted, network indices that are a plurality of features may be determined from the brain activity data.

According to another feature of the present disclosure, after the step S230 in which the features are extracted, main data having a high contribution to classification of a major depressive disorder or normal, that is, main features may be determined.

In this case, the main data may be determined based on statistical scores for the plurality of features that are obtained as a result of the step S230 in which the features are extracted.

For example, an independent sample t-test is performed on the network index for a brain active area determined in the step S230 in which the features are extracted, and features showing a significant difference depending on whether a major depressive disorder is present are determined. Then, after the Fisher's scores are calculated again on the features having a significant difference, the main data (or main feature) having a high contribution to classifying the major depressive disorder may be selected by ordering them. Meanwhile, determination of the main data is not limited to the above, and may be performed by more various statistical scoring methods.

Referring back to FIG. 2, in the step S240 in which whether the individual's major depressive disorder is present is determined, whether the individual has a major depressive disorder is determined based on the brain activity data and further, the features extracted from the brain activity data.

According to a feature of the present disclosure, in the step S240 in which whether the individual's major depressive disorder is present is determined, the classification model outputs whether the individual has a major depressive disorder by taking main data with a high contribution to classifying it as a major depressive disorder or normal as an input.

For example, in the step S240 in which whether the individual's major depressive disorder is present is determined, the classification model may output whether the major depressive disorder has occurred by taking at least one of theta strength, alpha strength, a theta clustering coefficient, and an alpha clustering coefficient for a right isthmus of cingulate area as an input. In addition, the classification model may output whether a major depressive disorder has occurred by taking at least one of a delta strength, an alpha strength, and an alpha clustering coefficient of a left postcentral area as an input.

Therefore, when feature parameters for all brain activity data are used, the classification model of the present disclosure can solve a problem of overfitting that appears in the model, reflect cognitive characteristics according to individuals, and allow for high-accuracy classification for a major depressive disorder.

According to a feature of the present disclosure, in the step S240 in which whether the individual's major depressive disorder is present is determined, whether the individual has a major depressive disorder may be determined according to an output result of the classification model which is further configured to output 0 or 1 depending on whether the individual has a major depressive disorder.

For example, referring together with FIG. 3D, in the step S240 in which whether the individual's major depressive disorder is present is determined, the classification model may output 1 when a risk degree of occurrence of the major depressive disorder for the individual is high based on the main data (or main feature) determined from the individual's brain activity data, and may output 0 when a probability of normal is high, in which the risk degree of occurrence is low.

The classification model may be further configured to output 0 or 1 depending on whether the major depressive disorder is present based on Equation 1 below.

$$Output = [ \frac{1}{n} {\sum }_{i = 1}^{n} max ( 0 , 1 - {y}_{i} ( ω \cdot {x}_{i - b} ) ) ] + λ {‖ ω ‖}^{2} \qquad [ Equation 1 ]$$

Hinge loss function, max(0,1−yᵢ(ω·x_{i-b}))
xᵢ=features for class 1 (MDD patients)
yᵢ=features for class 2 (healthy controls)
output 1 or 0 {1—true, 0—false}

Here, xᵢ is a feature value of an i-th major depressive disorder individual, yᵢ is a feature value of an i-th normal individual, ω refers to a weight, λ refers to a regularization coefficient, and b refers to a constant. In this case, the constant b may be determined through calculation of a hyperplane.

Accordingly, a user or medical team may confirm whether a major depressive disorder has occurred according to the output result (0 or 1).

As a result of the step S240 in which whether the individual's major depressive disorder is present is determined, pieces of information related to the major depressive disorder for the individual may be determined, and finally, in the step S250 in which the result is provided, various pieces of information determined by the classification model may be output or may be transmitted to the user mobile device, the medical team device, or the like.

Meanwhile, according to another feature of the present disclosure, when the risk degree of occurrence of the major depressive disorder for the individual is determined, according to a treatment progress, steps of receiving brain wave data, generating brain activity data, and re-determining whether the individual's major depressive disorder is present may be repeatedly performed.

By the method for providing information on a major depressive disorder according to various embodiments of the present disclosure, users can easily obtain information on their own mental health without temporal and spatial restrictions. Moreover, the medical team can obtain information on the individual, so that continuous monitoring such as evaluation of treatment prognosis for an individual who is suspected of a major depressive disorder may be enabled.

Evaluation: Feature Extraction for Major Depressive Disorder Classification and Classification Performance Evaluation of Device for Providing Information on Major Depressive Disorder.

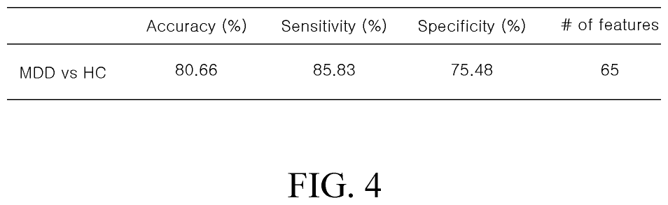

Hereinafter, an evaluation result of the device for providing information on a major depressive disorder according to an embodiment of the present disclosure will be described with reference to FIG. 4.

In this evaluation, brain wave data for a total of 50 individuals with major depressive disorders (MDDs) and 50 normal individuals in a control group (healthy control, HC) were used.

More specifically, according to another feature of the present disclosure from the brain wave data in this evaluation, the brain activity data were obtained for a total of 68 regions of interest (ROIs) of left and right brains regarding banks of the superior temporal sulcus, caudal anterior cingulate, caudal middle frontal, cuneus, entorhinal, frontal pole, fusiform, inferior parietal, inferior temporal, insula, isthmus cingulate, lateral occipital, lateral orbito frontal, lingual, medial orbito frontal, middle temporal, para central, para hippocampal, pars opercularis, pars orbitalis, pars triangularis, pericalcarine, post central, posterior cingulate, precentral, precuneus, rostral anterior cingulate, rostral middle frontal, superior frontal, superior parietal, superior temporal, supramarginal, temporal pole, and transverse temporal. Then, a connectivity of a phase locking value (PLV) was determined, and based on strength and a clustering coefficient of the connectivity of the PLV, a plurality of features were determined and finally, according to Fisher's score calculation result, 65 features were determined. Then, the classification model classified whether a major depressive disorder is present (0 or 1) for a major depressive disorder group or a control group based on 65 features, and accuracy, sensitivity, and specificity of the classification result were evaluated.

According to the evaluation result, based on the classification model, the accuracy of classification is 80.66%, the sensitivity is 85.83%, and the specificity is 75.48%.

This result may mean that the classification model classifies and provides whether the major depressive disorder has occurred in the individual with high reliability.

Accordingly, the present disclosure can overcome limitations of a fMRI-based diagnostic system for a major depressive disorder, the fMRI-based diagnostic system providing low-reliable information because it only focuses on neural activity while processing emotional information and does not consider important pathologies such as altered cognitive processes, and the fMRI-based diagnostic system still having many limitations such as involving expensive analysis costs, spatial and temporal restrictions, and the like.

In addition, the present disclosure allows users to easily obtain information on their own mental health without temporal and spatial restrictions, and a medical team can obtain information on an individual, so that continuous monitoring of an individual suspected of a major depressive disorder may be enabled.

Therefore, the present disclosure can contribute to an early diagnosis and a good treatment prognosis of a major depressive disorder by providing information on whether the major depressive disorder has occurred.

Although the embodiments of the present disclosure have been described in detail with reference to the accompanying drawings, it is to be understood that the present disclosure is not limited to those embodiments and various changes and modifications may be made without departing from the scope of the present disclosure. Therefore, the embodiments disclosed in the present disclosure are intended to illustrate rather than limit the scope of the present disclosure, and the scope of the technical idea of the present disclosure is not limited by these embodiments. Therefore, it should be understood that the above-described embodiments are illustrative in all aspects and not restrictive. The scope of the present disclosure should be construed according to the claims, and all technical ideas in the scope of equivalents should be construed as falling within the scope of the present disclosure.

[National R&D projects that supported this invention]

[Assignment unique number] 2018R1A2A2A05018505

[Department name] Ministry of Science and ICT

[Research Management Professional Institution] National Research Foundation of Korea

[Research Project Name] Mid-level researcher support project

[Research Study Name] Development of prediction and diagnostic tool for mental illness using EEG and HRV and machine learning

[Contribution rate] 1/1

[Organizing Agency] Inje University (Medical school)

[Study period] 20200301-20210228

1. A method for providing information on a major depressive disorder, implemented by a processor, the method comprising:
receiving brain wave data of an individual;
generating brain activity data based on the brain wave data; and
determining whether the individual's major depressive disorder is present by using a classification model configured to classify the major depressive disorder based on the brain activity data.

2. The method of claim 1, further comprising:
after generating the brain activity data,
extracting a feature of the brain activity data,
wherein the determining of whether the major depressive disorder is present includes determining whether the individual's major depressive disorder is present based on the feature, by using the classification model.

3. The method of claim 2, wherein the brain activity data is a plurality of pieces of brain activity data,
wherein the extracting of the feature includes,
determining a functional connectivity between the plurality of pieces of brain activity data, and
determining the feature of the brain activity data based on a network structural characteristic of the functional connectivity.

4. The method of claim 3, wherein the determining of the functional connectivity includes,
determining a connectivity of a phase locking value (PLV) for each of the plurality of pieces of brain activity data,
wherein the determining of the feature of the brain activity data includes,
determining the feature based on strength and a clustering coefficient of the connectivity of the PLV for the each of the plurality of pieces of brain activity data.

5. The method of claim 1, wherein the classification model is further configured to output 0 or 1 depending on whether the individual's major depressive disorder is present,
wherein the determining of whether the major depressive disorder is present includes
determining whether the individual's major depressive disorder is present according to a result of the output.

6. The method of claim 1, wherein the brain activity data is a plurality of pieces of brain activity data,
wherein the method further includes determining main data having a significant difference depending on whether the major depressive disorder is present among the plurality of pieces of brain activity data,
wherein the determining of whether the major depressive disorder is present includes determining whether the major depressive disorder is present based on the main data, by using the classification model.

7. The method of claim 6, wherein the determining of the main data includes,
extracting a feature of each of the brain activity data; and
determining the main data based on a statistical scoring method for the feature.

8. The method of claim 6, wherein the main data includes,
brain activity data of a right isthmus of cingulate, and brain activity data of a left postcentral area.

9. The method of claim 8, wherein the brain activity data of the right isthmus of cingulate is at least one of theta strength, alpha strength, a theta clustering coefficient, and an alpha clustering coefficient,
wherein the brain activity data of the left postcentral area is at least one of delta strength, alpha strength, and an alpha clustering coefficient.

10. The method of claim 1, further comprising:
filtering the brain activity data based on a band pass filter, which is performed after the generating of the brain activity data,

11. The method of claim 1, wherein the brain wave data is defined as brain wave data obtained in a resting state.

12. The method of claim 1, wherein the generating of the brain activity data includes converting the brain wave data into the brain activity data, by using at least one among low-resolution brain electromagnetic tomography (LORETA), standardized low-resolution brain electromagnetic tomography (sLORETA), exact resolution brain electromagnetic tomography (eLORETA), minimum-norm estimate (MNE) and dynamic statistical parametric mapping (dSPM).

13. The method of claim 1, wherein the brain activity data includes a current source density (CSD) in at least one brain area among banks of the superior temporal sulcus, caudal anterior cingulate, caudal middle frontal, cuneus, entorhinal, frontal pole, fusiform, inferior parietal, inferior temporal, insula, isthmus cingulate, lateral occipital, lateral orbito frontal, lingual, medial orbito frontal, middle temporal, para central, para hippocampal, pars opercularis, pars orbitalis, pars triangularis, pericalcarine, post central, posterior cingulate, precentral, precuneus, rostral anterior cingulate, rostral middle frontal, superior frontal, superior parietal, superior temporal, supramarginal, temporal pole, and transverse temporal.

14. The method of claim 1, further comprising:
when a risk of occurrence of the major depressive disorder for the individual is determined,
according to a treatment progress,
repeatedly performing
the receiving of the brain wave data;
the generating of the brain activity data; and
the determining of whether the individual's major depressive disorder is present.

15. A device for providing information on a major depressive disorder, the device comprising:
a receiver configured to receive brain wave data of an individual; and
a processor coupled to the receiver to communicate therewith,
wherein the processor is further configured to generate brain activity data based on the brain wave data, and determine whether the individual's major depressive disorder is present by using a classification model configured to classify a major depressive disorder based on the brain activity data.

16. The device of claim 15, wherein the processor is further configured to extract a feature of the brain activity data and determine whether the individual's major depressive disorder is present based on the feature, by using the classification model.

17. The device of claim 16, wherein the brain activity data is a plurality of pieces of brain activity data,
wherein the processor is further configured to determine a functional connectivity between the plurality of pieces of brain activity data and determine the feature of the brain activity data based on a network structural characteristic of the functional connectivity.

18. The device of claim 17, wherein the processor is further configured to determine a connectivity of a phase locking value (PLV) for each of the plurality of pieces of brain activity data and determine the feature based on strength and a clustering coefficient of the connectivity of the PLV for the each of the plurality of pieces of brain activity data.

19. (canceled)

20. The device of claim 15, wherein the brain activity data is a plurality of pieces of brain activity data, and
the processor is further configured to determine main data having a significant difference depending on whether the major depressive disorder is present among the plurality of pieces of brain activity data, and determine whether the major depressive disorder is present based on the main data, by using the classification model.

21. The device of claim 20, wherein the main data includes brain activity data of a right isthmus of cingulate, and brain activity data of a left postcentral area,
wherein the brain activity data of the right isthmus of cingulate is at least one of theta strength, alpha strength, a theta clustering coefficient, and an alpha clustering coefficient, and
wherein the brain activity data of the left postcentral area is at least one of delta strength, alpha strength, and an alpha clustering coefficient.

22-24. (canceled)
